(19) United States
(12) Patent Application Publication
(10) Pub. No.: US 2010/0298873 A1
(43) Pub. Date: Nov. 25, 2010

(54) SURGICAL SUTURE MATERIAL WITH ANCHORING ELEMENTS

(75) Inventors: Erich Odermatt, Schafhausen (CH); Silke Konig, Rottweil (DE)

Correspondence Address: IP GROUP OF DLA PIPER LLP (US), ONE LIBERTY PLACE, 1650 MARKET ST, SUITE 4900, PHILADELPHIA, PA, 19103, US

(73) Assignee: AESCULAP AG, Tuttlingen/Donau (DE)

(21) Appl. No.: 12/812,318

(22) PCT Filed: Jan. 9, 2009
(86) PCT No.: PCT/EP2009/000082

§ 371 Date: Jul. 9, 2010

(30) Foreign Application Priority Data
Jan. 9, 2008 (DE) 10 2008 004 574.8

(51) Int. Cl. A61B 17/04

(52) U.S. Cl. 606/228

(57) ABSTRACT

A surgical suture material having a surface with anchoring elements made of a shape-memory polymer.

### RELATED APPLICATIONS

This is a §371 of International Application No. PCT/EP2009/000082, with an international filing date of Jan. 9, 2009 (WO 2009/087105 A1, published Jul. 16, 2009), which is based on German Patent Application No. 10 2008 004 574.8, filed Jan. 9, 2008, the subject matter of which is incorporated by reference.

### TECHNICAL FIELD

This disclosure relates to a surgical suture material with anchoring elements on its surface, to its use in surgery, and to a surgical kit.

### BACKGROUND

In skin closure, for example, in plastic surgery, adverse skin reactions often occur, especially at the point where the suture is knotted. This can lead to unsatisfactory cosmetic results for the patients concerned. Wounds should in principle be sutured with a certain pressure at the wound margins. If the wound margins are sutured too loosely and too irregularly, there is in principle a risk of increased scar formation. By contrast, if the wound margins are sutured too strongly, there is a danger of the circulation of blood in the wound margins being restricted, which can lead to necrotic changes in the surrounding tissue area.

In recent years, therefore, suture materials have increasingly been developed that permit wound closure without knots. Such suture materials have become known as barbed sutures. These are suture materials that have barbs protruding from their surfaces. The barbs are designed to fix the suture material in the tissue. To be able to fix the suture material adequately in tissue, the barbs are normally stiff structures. However, the stiffness of the barbs increases the resistance that has to be overcome when pulling the suture material into a wound area to be treated. As a result, wound treatment with the aid of such suture materials can cause undesired tissue damage.

It could therefore be helpful to provide a surgical suture material which allows wounds to be closed without knots and in a manner that is gentle on the tissue. The suture material should also be as easy to handle as possible and avoid the disadvantages known from the prior art.

### SUMMARY

We provide a surgical suture material having a surface with anchoring elements made of a shape-memory polymer.

We also provide a method of closing skin in plastic surgery including applying the surgical suture material to adjacent portions of skin and thereby drawing the adjacent portions into substantial contact.

We further provide a method of suturing adjacent portions of tissue in abdominal or gynecological surgery including applying the surgical suture material to the adjacent portions and thereby drawing the adjacent portions into substantial contact.

We also further provide a surgical kit including at least one surgical needle and the suture material.

### BRIEF DESCRIPTION OF THE DRAWINGS

Further features and details will be come clear from the following description of the figures. Individual features can be realized either singly or severally in combination. By express reference, the figures are herewith made part of the content of the description.

In the schematic figures:

FIG. 1 shows a suture material in a temporary shape.

FIG. la shows a cross section of the suture of FIG. 1.

FIG. 2 shows a suture material from FIG. 1 in a permanent shape.

FIG. 2a shows a cross section of the suture of FIG. 2.

FIG. 3 shows various parameters for characterizing the anchoring elements.

### DETAILED DESCRIPTION

We provide a surgical suture material which, on its surface, has anchoring elements made of a shape-memory polymer.

A suture material is made available for anchoring, preferably self-anchoring or knotless anchoring, in biological tissues, particularly human and/or animal tissues, wherein the anchoring elements or anchoring structures provided are made of a shape-memory polymer. The tissues can be, for example, skin, fat, fascias, bones, muscles, organs, nerves, blood vessels, connective tissues, tendons or ligaments. In this way, the shape-changing properties of shape-memory polymers can be advantageously exploited to perform uncomplicated, knotless wound treatment, in particular, wound closure, especially in a manner that is gentle on tissue. The anchoring structures themselves are preferably formed by incisions into the suture material.

Preferably, the anchoring elements in an unimplanted state of the suture material are formed bearing on the surface thereof, preferably bearing closely or tightly thereon. The anchoring elements preferably do not protrude substantially from the suture material surface. This usually represents the so-called “temporary” state of the suture material. The anchoring elements preferably bear closely on the suture material surface in such a way that the surface appears smooth, at least on macroscopic observation. This has the advantage that the suture material can be pulled into a wound area to be treated without any appreciable resistance from the anchoring elements.

Anchoring elements produced by incisions into the suture material each may enclose an angle α of between 120 and 175° , in particular, of between 140 and 160° , with the suture material surface.

The anchoring elements can preferably be converted to a shape in which they protrude from the suture material surface. In principle, the anchoring elements of the suture material can be converted by suitable stimuli to a shape in which they protrude from the suture material surface. The stimuli can, in particular, be physical and/or chemical stimuli. The physical stimuli can be thermal, optical, electric and/or magnetic stimuli, for example. Suitable chemical stimuli are, for example, changes in ionic strength and/or pH value. The aforementioned thermal stimulus is preferably the human body temperature.

The anchoring elements can preferably be converted by a change in temperature, in particular, an increase in temperature, to a shape in which they protrude from the suture material surface. The anchoring elements can preferably be converted in a temperature range of between 30 and 42° C., in particular, of between 35 and 40° C., to a shape in which they protrude from the suture material surface. The anchoring elements can particularly preferably be converted to the protruding shape at the body temperature of a patient. This has the particular advantage that the anchoring elements are able to set themselves upright independently after implantation, in particular after subcutaneous implantation, of the suture material.

The anchoring elements can in principle have any desired shapes. For example, the anchoring elements can be in the form of hooks, barbs, arrows, rods, escutcheons, scales, shields, wedges or the like. Moreover, the anchoring elements can also be V-shaped and/or W-shaped. It is particularly preferable if the anchoring elements are designed in the manner of barbs.

The anchoring elements can in principle be formed in different arrangements on the surface of the suture material. For example, the barbs can have a row by row arrangement, an offset arrangement, a zigzag arrangement, a spiral-shaped arrangement, a random arrangement, or combinations of these, in the longitudinal and/or transverse direction, preferably in the longitudinal direction, of the suture material. The anchoring elements can in particular be arranged in one or more rows and/or as helices on the suture material. An arrangement may also be preferred in which the anchoring elements are distributed across the entire surface of the suture material. This permits a particularly secure anchoring of the suture material in a surrounding tissue area.

The suture material may have at least one set, in particular two, three or more sets, of anchoring elements. A set of anchoring elements is to be understood here as an arrangement of anchoring elements, on the surface of the suture material, that corresponds in respect of the configuration of the anchoring elements, for example, in respect of the height of the anchoring elements, the length of the anchoring elements, the angle which the anchoring elements form with the surface of the suture material and/or the shape of the anchoring elements.

The suture material particularly preferably has what is called a “bidirectional arrangement of anchoring elements.” A bidirectional arrangement of anchoring elements is to be understood as an arrangement in which the anchoring elements are oriented in two different directions. Preferably, seen in the longitudinal direction of the suture material, the anchoring elements for a first suture material portion are preferably formed in the direction of another, second suture material portion, and the anchoring elements for the other, second suture material portion are formed in the direction of the first suture material portion. Particularly preferably, seen in the longitudinal direction of the suture material, the anchoring elements for a first suture material portion are oriented in the direction of the center of the suture material and, for another, second suture material portion, are likewise oriented in the direction of the center of the suture material. The length of the suture material portions preferably corresponds approximately to half the suture material length, such that the suture material center forms a kind of center of symmetry. In this way, the suture material can be pulled from one end thereof to approximately the center of the length of the suture material through a biological tissue, without any great resistance, and, when a pull is exerted in the opposite direction, the anchoring elements preferably stand upright and in this way anchor or fix the suture material in the tissue, without knots being needed.

The surgical suture material may have at least two bidirectional arrangements of anchoring elements on its surface. It is particularly preferable if, in relation to a first bidirectional arrangement of anchoring elements, a second bidirectional arrangement of anchoring elements is formed on the suture material surface at approximately 180° in the circumferential direction of the suture material and preferably offset in relation to the first bidirectional arrangement. It is also possible for the surgical suture material to have a total of three bidirectional arrangements of anchoring elements. In this case, it is preferable if, in relation to a first bidirectional arrangement of anchoring elements, a second bidirectional arrangement of anchoring elements is formed on the suture material surface at approximately 120° in the circumferential direction of the suture material and preferably offset in relation to the first bidirectional arrangement, which second bidirectional arrangement of anchoring elements is in turn formed at approximately 120° in the circumferential direction of the suture material and preferably offset in relation to a third bidirectional arrangement of anchoring elements, such that the third bidirectional arrangement of anchoring elements is likewise formed at approximately 120° in the circumferential direction of the suture material and preferably offset in relation to the first bidirectional arrangement of anchoring elements.

The anchoring elements are normally formed in one piece with the suture material.

The anchoring elements can have a thickness of between 50 and 1000 μm. The thickness of the anchoring elements is preferably between 100 and 500 μm. Moreover, the anchoring elements can have a length of between 100 and 2000 μm. The anchoring elements preferably have a length of between 250 and 1500 μm, in particular a length of ca. 1500 μm. The anchoring elements are present on the suture material surface in a density of 6 to 10 anchoring elements per 5 mm length of the suture material. The lengths described above are preferably cut lengths that can be produced by incisions made in the suture material.

For the shape-memory polymer in question, it is possible in principle to use all polymers with shape-memory properties. The shape-memory polymer is preferably a thermoplastic shape-memory polymer. The shape-memory polymers (SMPs) can also preferably be segmented copolymers, so-called “block” copolymers, preferably with a linear structure. Copolymers within the meaning of this disclosure are to be understood generally as polymers composed of at least two, in particular of two, three, four or more, different monomer types. The shape-memory polymers can be present as di-, tri-, tetra- or multi-block copolymers and generally have at least one crystalline hard segment and at least one amorphous soft segment.

The hard segments can generally be characterized on the basis of a melting point and the soft segments on the basis of a glass transition temperature Tₘ. For simplicity, the term transition temperature Tₜᵣₐₙₛ or restoring temperature Tᵣ is mostly used. The transition temperature Tₜᵣₐₙₛ or the restoring temperature Tᵣ is the temperature at which the shape-memory polymer returns to a previously programmed, permanent shape. Tₜᵣₐₙₛ or Tᵣ can be a glass temperature T_{g} of amorphous areas or a melting temperature Tₘ of crystalline areas of the shape-memory polymer. It is designated in general hereinbelow as Tₜᵣₐₙₛ and may vary depending on the composition and mixing ratio of segments of the shape-memory polymer.

If a thermoplastic shape-memory polymer is heated to a temperature above the transition temperature Tₜᵣₐₙₛ of the hard segment, the polymer can be shaped. The shape can be stored or programmed as what is called a permanent shape, by means of the shape-memory polymer being cooled to below the transition temperature Tₜᵣₐₙₛ of the hard segment. If the shape-memory polymer that has been shaped in this way is cooled to below the transition temperature Tₜᵣₐₙₛ of the soft segment, while the shape of the polymer is changed, a new, so-called “temporary” shape of the shape-memory polymer can be fixed. The permanent shape can be recovered by heating the shape-memory polymer through Tₜᵣₐₙₛ of the soft segment to Tₜᵣₐₙₛ or Tᵣ of the hard segment.

The shape-memory polymer can have varying hard segment and/or soft segment fractions. The shape-memory polymer is preferably a block copolymer with a hard-segment fraction of between 5 and 95% by weight, in particular of between 20 and 80% by weight. The shape-memory polymer, as block copolymer, preferably has a soft-segment fraction of between 95 and 5% by weight, in particular of between 80 and 20% by weight.

The shape-memory polymer may be a block copolymer with a hard-segment fraction whose transition temperature Tₜᵣₐₙₛ is at least 10 to 20° C. higher than the transition temperature Tₜᵣₐₙₛ of a soft segment also contained in the block copolymer. The shape-memory polymer is preferably a block copolymer with a hard-segment fraction whose transition temperature Tₜᵣₐₙₛ is between 10 and 250° C., in particular, between 30 and 200° C. The shape-memory polymer is preferably a block copolymer with a soft-segment fraction whose transition temperature Tₜᵣₐₙₛ is between 10 and 250° C., in particular, between 15 and 60° C., preferably between 25 and 50° C.

The shape-memory polymer may be a block copolymer that has a hard-segment fraction with a melting enthalpy of between 15 J/g and 500 J/g. The shape-memory polymer can have a degree of crystallinity of between 20 and 80%, in particular of between 30 and 70%. The shape-memory polymer can have a molecular weight of between 500 g/mol and 6,000,000 g/mol. In particular, hard and/or soft segments contained in the shape-memory polymer can have a molecular weight of between 20,000 g/mol and 600,000 g/mol.

The shape-memory polymer can in principle be a natural polymer, a so-called “biopolymer.” For example, the shape-memory polymer can be a protein or polysaccharide. Examples of proteins are zein, casein, gelatin, glutin, serum albumin and/or collagen. Suitable polysaccharides are chosen, for example, from the group including alginate, celluloses, dextrans, pullulan, hyaluronic acid, chitosan and chitin.

The shape-memory polymer can also be a modified biopolymer. These include cellulose derivatives, in particular, alkyl celluloses, hydroxyalkyl celluloses, cellulose ethers, cellulose esters, nitrocelluloses and chitosan. The alkyl celluloses can be, for example, methyl cellulose and/or ethyl cellulose. Examples of suitable hydroxyalkyl celluloses include hydroxyl-propyl cellulose, hydroxypropyl methyl cellulose and/or hydroxybutyl methyl cellulose. Other cellulose derivatives that can be used are cellulose acetate, cellulose propionate, cellulose acetate butyrate, cellulose acetate terephthalate, carboxymethyl cellulose, cellulose triacetate and/or cellulose sulfate salts.

The shape-memory polymer is preferably a synthetic polymer. Possible synthetic polymers are in principle resorbable and non-resorbable polymers. Possible synthetic non-resorbable polymers are, for example, polyphosphazenes, polyamides, polyester amides, polyanhydrides, polycarbonates, polyacrylates, polyalkylenes, polyacrylamides, polyalkylene glycols, polyalkylene oxides, polyalkylene terephthalates, polyorthoesters, polyvinyl ethers, polyvinyl esters, polyvinyl halides, polyvinyl pyrrolidones, polyesters, polysiloxanes, polyurethanes, mixtures thereof and/or copolymers thereof.

Suitable examples of non-resorbable polymers include, in particular, ethylene vinyl acetate, polyethylene, polypropylene, polystyrene, polyvinyl chloride, polyvinyl phenol, polymethyl methacrylate, polybutyl methacrylate, polyisobutyl methacrylate, polyhexyl methacrylate, polyisodecyl methacrylate, polylauryl methacrylate, polyphenyl methacrylate, polyhydroxypropyl methacrylate, polyethyleneglycol methacrylate, polymethyl acrylate, polyisopropyl acrylate, polyisobutyl acrylate, polyoctadecyl acrylate, polyhydroxyethyl acrylate, polyhydroxypropyl acrylate, polybutyl acrylate, mixtures thereof and/or copolymers thereof

Suitable resorbable polymers are, in particular, polyhydroxy acids, preferably polylactides, polyglycolides, polyhydroxybutyric acid, polyhydroxyvaleric acid, polylactide-co-glycolides, polylactide-co-ε-caprolactone, polyglycolide-co-ε-caprolactone, polyamino acids, poly-pseudoamino acids, polyhydroxylalkanoates, polyvinyl alcohols, mixtures thereof and/or copolymers thereof.

Provision can also be made for the shape-memory polymer to be produced from a polymer mixture or from a polymer blend containing any combination of the previously mentioned polymers.

The shape-memory polymer may form network structures. Such network structures can be produced by covalent crosslinking of suitable macromonomers, i.e., polymers or oligomers with polymerizable end groups. The polymerization is normally induced by the influence of ultraviolet light or by means of a suitable polymerization initiator.

The shape-memory polymer can, in particular, be present in the form of two mutually penetrating networks. These are usually networks in which two polymer components are crosslinked, but not with each other. In this case, the original or permanent shape of the shape-memory polymer is generally determined by the network with the highest crosslinking density and the highest mechanical strength. Moreover, the shape-memory polymer in this case usually has two different transition temperatures that correspond to different soft segments of both networks.

Moreover, the shape-memory polymer of the suture material can be present in the form of mixed, mutually penetrating networks. Such networks generally comprise at least one physically crosslinked polymer network, usually on the basis of a thermoplastic polymer, and at least one covalently crosslinked polymer network, normally based on a thermoset polymer. The two polymer components cannot normally be separated from each other by physical processes. The permanent shape is fixed by the covalently crosslinked network. The permanent shapes are determined by the transition temperatures of soft segments of the thermoplastic polymer and of the thermoset polymer and also by the transition temperature of a hard segment of the thermo-plastic polymer.

It is also possible for the shape-memory polymer to be in the form of semi-penetrating networks. Such networks are normally defined as two mutually independent components, of which one component is a crosslinked polymer and the other component is a non-crosslinked polymer. Again, the components cannot generally be separated from each other by physical processes. The semi-penetrating networks usually have at least one thermal transition, which corresponds to at least one soft segment of the non-crosslinked polymer.

Suitable network structures can be constructed from, for example, poly-(ε-caprolactone) dimethyl acrylate and n-butyl acrylate, polyethylene terephthalate and polyethylene oxide or from polystyrene and poly-1,4-butadiene.

The shape-memory polymer preferably forms a photosensitive polymer network. Such a network usually has a matrix based on polyacrylates and/or polymethacrylates, in particular, the aforementioned polybutyl acrylate and polyhydroxyethyl methacrylate. In addition to the matrix, the network usually also has a crosslinking agent and a photoreactive component. The crosslinking agents can be bifunctional or polyfunctional crosslinking agents, in particular, oligomeric, linear diacrylate crosslinking agents, for example, poly(oxyethylene) diacrylates or poly(oxypropylene) diacrylates. Photoreactive components that can be used are, in particular, cinnamic esters. Thus, it is known that cinnamic acid and derivatives thereof dimerize to cyclobutane compounds under the influence of ultraviolet light with a wavelength of approximately 300 nm. The photoreaction is reversible. The dimers can therefore be cleaved again. For this purpose, the dimer compounds are usually irradiated with ultraviolet light with a shorter wavelength, for example, of approximately 240 nm. By a suitable choice of substituents on the phenyl ring of the cinnamic acid, the absorption maxima can be shifted within the UV range. Normally, the photoreactive component is polymerized into the network matrix or is mixed with the network matrix by physical processes, in particular, in the manner of a mutually penetrating network.

In accordance with the observations made in the preceding paragraph, the principle by which a photosensitive network functions can be described as follows.

The network typically has a permanent shape. Upon deformation of the network and irradiation with ultraviolet light of a suitable wavelength, the photoreactive components contained in the network form covalent bonds with one another. In addition, the network is preferably crosslinked by the crosslinking agents contained therein. A temporary shape of the network is programmed in this way. Since the photo-crosslinking is reversible, renewed irradiation with light of another wavelength makes it possible to undo the crosslinking and recover the permanent shape of the network.

The suture material can be a monofilament and/or multifilament material, in particular, a monofilament material. In the case of a multifilament suture material, the anchoring elements can be individual threads of the multifilament. The suture material can also be braided or twined. The suture material can also have the thread strengths typical of suture materials, in particular, thread strengths of between USP 8/0 and USP 6. In the case of monofilament suture materials, the thread strengths are preferably between USP 4/0 and USP 2, in particular USP 2/0.

Preferably, the suture material is formed from the same shape-memory polymer as the anchoring elements on its surface. Therefore, as regards the shape-memory polymers in question, reference is made to the whole of the previous description.

It may also be preferable for the suture material to be coated, in particular, with a lubricant layer that is resorbable in body fluids. A particular advantage lies in improved protection against possible tissue trauma during introduction of the suture material into a biological tissue. Depending on the nature of the coating, the latter results in a certain degree of adherence of the suture material in the tissue concerned, such that the anchoring or fixing of the suture material in the tissue can be additionally improved in this way.

Provision can also be made for the suture material to comprise active substances, in particular, antimicrobial, disinfecting, anti-inflammatory, growth-promoting, deodorizing and/or analgesic active substances.

At least one end of the suture material may be connected to a surgical needle. It may be preferable for both ends of the suture material to be connected to a respective surgical needle. To connect the suture material to a surgical needle, the thread is generally introduced into a needle bore provided for this purpose, and the needle is then pressed together or crimped in the area of the bore.

The suture material may be present in a sterilized and in particular packaged form.

A further aspect concerns a surgical kit or set comprising at least one surgical needle and a suture material. The kit or set can, in particular, comprise two surgical needles. For further features and details of the kit or set, reference is made to the above description.

We finally provide for the use of the suture material as a self-fixing suture material, in particular, without knots. The suture material is particularly suitable for indications in which the cosmetic result is especially important to the patient. Therefore, a further aspect concerns the use of the suture material in plastic surgery, in particular, for closing skin, preferably for closing facial skin. A further application in the field of plastic surgery concerns the use of the suture material for tightening the skin, for example, for eyebrow lifts. Moreover, the suture material is also suitable for treatment of internal wounds, in particular, wounds in the abdominal area, and wounds that are difficult to access by laparoscopy. Moreover, the suture material can also be used for fixing implants, in particular, meshes, for example, hernia meshes, prolapse meshes or urinary incontinence meshes. The suture material is preferably used in abdominal and/or gynecological surgery. A further possible area of use of the suture material is in the formation of anastomoses, in particular, vascular or intestinal anastomoses.

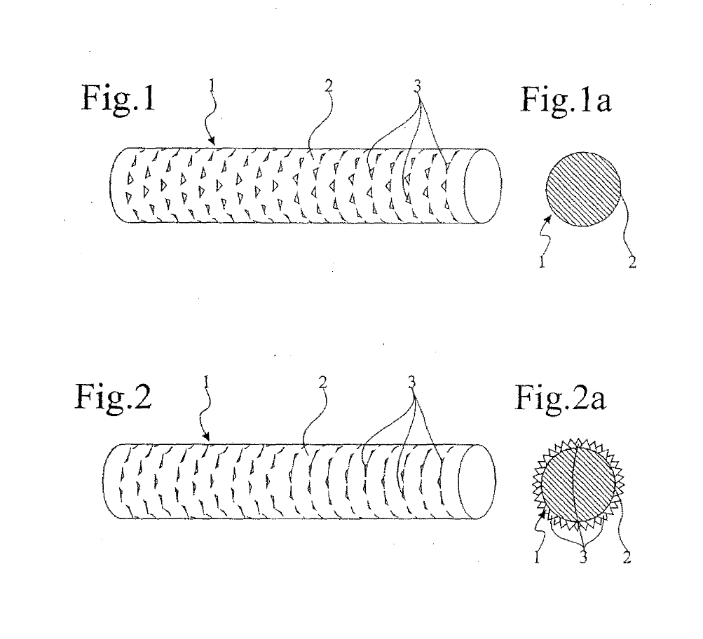

Turning now to the drawings, the temporary shape of a suture material 1 made of a thermoplastic shape-memory polymer is shown schematically in FIG. 1. The suture material 1 has barb-shaped or spike-shaped anchoring elements 3 on its surface 2. Starting from half way along the length of the suture material 1, these anchoring elements 3 point in opposite directions. The anchoring elements 3 can be produced, for example, by incisions made in a suture material made of a shape-memory polymer. The anchoring elements 3 bear closely on the suture material surface 2 in such a way that the surface 2 appears substantially smooth on the outside (see FIG. 1a). The anchoring elements bearing closely on the surface offer no resistance or only very slight resistance in the direction of pulling through, such that tissue trauma can be avoided. Counter to the direction of pulling through, the suture material 1 offers a sufficient holding force in the tissue to ensure that the approximation of the wound margins can take place substantially without tissue trauma.

FIG. 2 is a schematic representation of the suture material 1 described in FIG. 1, now in the so-called “permanent” shape. In this shape, the barb-shaped or spike-shaped anchoring elements 3 protrude from the suture material surface 2 (see also FIG. 2a). This can be brought about, for example, by the body temperature of a patient after implantation of the suture material. In this way, the anchoring elements 3 lift and are converted from the shape shown in FIG. 1 to a shape in which they protrude from the suture material surface 2. At the same time, the suture material contracts on account of heating to body temperature. The anchoring elements 3 engage and exert a certain pressure on the wound margins and press these relatively smoothly together.

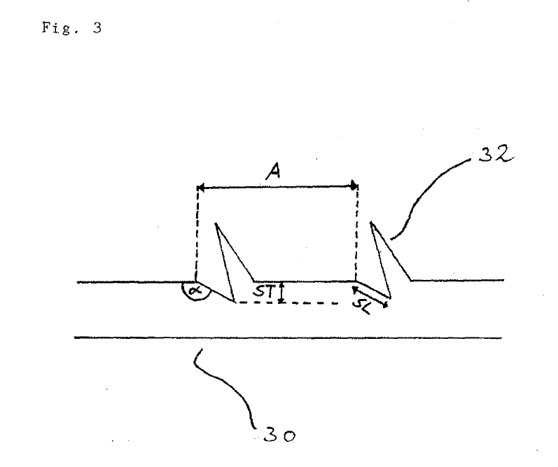

FIG. 3 is a schematic representation of a side view of a suture material 30 with two anchoring elements 32 in the form of barbs. The anchoring elements 32 can have a certain distance A from each other. This distance can be between 250 and 1500 μm, for example. Further parameters or variables for the anchoring elements 32 are the angle α, the cutting depth ST, and the cutting length SL. The latter are related as follows:

SL=ST/sin(180°−α).

### EXAMPLES

### Example 1

A polymer network with shape-memory properties, based on methacrylate-terminated ((ε-hydroxycaproate)-co-glycolate)diol oligomers, methacrylate-terminated ((ε-hydroxycaproate)-co-glycolate)diol oligomers and butyl acrylate as comonomer or oligo(p-dioxanone)diol and crystallizable oligo(p-dioxanone)diol, or a copolyester-urethane network with a shape-memory effect is extruded to form a thread. After extrusion, the thread, still in the warm state (e.g., 37° C.), is incised or worked with similarly warm knives or blades or the like in one direction, or in two opposite directions starting from the center (permanent shape). The spikes are thus introduced into the thread. The incised thread is then drawn through or immersed in a cooled (25° C.) tube system or hollow system or press system. The hooks settle and, by means of the cooling and the shape-memory effect, the hooks are fixed bearing on the thread (temporary shape).

### Example 2

A thread material made of a shape-memory polymer is made available for various types of fixing on or in bone or cartilage. Two or more incisions are made in the end of the thread material, for example, under heat. The resulting protruding hooks are pressed down with cooling and are thus fixed bearing on the surface.

### Example 3

A thread material is made available with an “umbrella shape” or a kind of “anchor” at one end or at both ends, for example, to fix a surgical mesh to the tissue. The cut for the “umbrella shape” is introduced with heat around one end or both ends of the thread material by means of, for example, a blade or a knife with a round or roundish shape and with the aid of a laser. Alternatively, an “anchor shape” can be cut in at one end or both ends of the thread material or of a thicker thread. The “umbrella shape” or “anchor shape” is likewise fixed with cooling by pressing down of the protruding shape or by drawing into a cooled tube system.

After the thread or thread materials described in Examples 1 to 3 have been implanted in a human or animal body, the hooks stand upright because of the shape-memory effect and in this way prevent the implanted threads or thread materials from slipping.

1-21. (canceled)

22. A surgical, suture material, having a surface with anchoring elements made of a shape-memory polymer.

23. The surgical suture material as claimed in claim 22, wherein, in an unimplanted state, the anchoring elements are formed bearing on the surface thereof.

24. The surgical suture material as claimed in claim 22, wherein the anchoring elements can be converted to a shape in which they protrude from the suture material surface.

25. The surgical suture material as claim, in claim 24, wherein the anchoring elements are converted by chemical and/or physical stimulus.

26. The surgical suture material as claimed in claim 22, wherein the anchoring elements, produced by incisions into the suture material, each enclose an angle a of between 120 and 175° with the suture material surface.

27. The surgical suture material as claimed in claim 22, wherein the anchoring elements can be converted by a change in temperature to a shape in which they protrude from the suture material surface.

28. The surgical suture material as claimed in claim 27, wherein the change in temperature is an increase in temperature.

29. The surgical suture material as claimed in claim 22, wherein the anchoring elements can be converted in a temperature range of between 30 ,and 42° C. to a shape in which they protrude from the suture, material surface.

30. The surgical suture material as claimed in claim 22, wherein the anchoring elements have a thickness of between 50 and 1000 μm.

31. The surgical suture material as claimed in claim 22, wherein the anchoring elements have a length of between 100 and 2000 μm.

32. The surgical suture material as claimed in claim 22, wherein the anchoring elements are present on the suture material surface in a density of 6 to 10 anchoring elements per 5 mm length of the suture material.

33. The surgical suture material as claimed in claim 22, wherein the shape-memory polymer is a block copolymer with a hard-segment fraction of between 5 and 95% by weight.

34. The surgical suture material as claimed in claim 22,, wherein the shape-memory polymer is present as a block copolymer with a soft-segment fraction of between 95 and 5% by weight.

35. The surgical suture material as claimed in claim 22, wherein the shape-memory polymer is a block copolymer with a hard-segment fraction whose transition temperature Tₜᵣₐₙₛ is at least 10 to 20° C. higher than the transition temperature Tₜᵣₐₙₛ of a soft segment also contained in the block copolymer.

36. The surgical suture material, as claimed in claim 22, wherein the shape-memory polymer is a block copolymer with a hard-segment fraction, whose transition temperature Tₜᵣₐₙₛ is between 10 and 250° C.

37. The. surgical suture material as claimed in claim 22, wherein the shape-memory polymer is a block copolymer with a soft-segment fraction whose transition temperature Tₜᵣₐₙₛ is between 10 and 250° C.

38. The surgical suture material as claimed in claim 22, wherein the shape-memory polymer is a block copolymer that has a hard-segment fraction with a melting enthalpy of between 15 and 500 J/g.

39. The surgical suture material as claimed in claim 22, wherein the shape-memory polymer has a degree of crystallinity of between 20 and 80%.

40. The surgical suture material as claimed in claim 22, wherein it is coated.

41. The surgical suture material as claimed in claim 40, wherein the material is coated with a lubricant layer resorbable in body fluids.

42. The surgical suture material as claimed in claim 22, which is self-fixing.

43. A method of closing skin in plastic surgery comprising applying the surgical suture material of claim 22 to adjacent portions of skin and thereby drawing the adjacent portions into substantial contact.

44. A method of suturing adjacent portions of tissue in abdominal or gynecological surgery comprising applying the surgical material of claim 22 to the adjacent portions and thereby drawing the adjacent portions into substantial contact

45. A surgical kit comprising at least one surgical needle, and a suture material as claimed in claim 22.
